(19) United States
(12) Patent Application Publication
(10) Pub. No.: US 2007/0270651 A1
(43) Pub. Date: Nov. 22, 2007

(54) Device and method for illuminating an in vivo site

(76) Inventors: Zvika Gilad, Haifa (IL); Elisha Rabinovitz, Haifa (IL)

Correspondence Address: PEARL COHEN ZEDEK LATZER, LLP, 1500 BROADWAY 12TH FLOOR, NEW YORK, NY, 10036, US

(21) Appl. No.: 11/802,121

(22) Filed: May 21, 2007

Related U.S. Application Data
(60) Provisional application No. 60/801,385, filed on May 19, 2006.

(51) Int. Cl. A61B 1/06

(52) U.S. Cl. 600/160

(57) ABSTRACT

The invention relates to a device and method for providing images of an in vivo site during in vivo procedures, such as laparoscopy wherein the device is capable of illuminating an internal body cavity and has grasping tongs for fastening to an internal body structure.

### CROSS-REFERENCE TO RELATED APPLICATIONS

This application claims benefit of U.S. Provisional Patent Applications Ser. No. 60/801,385, entitled “Device and method for immobilizing an in vivo capsule” filed May 19, 2006, the entire contents of which is incorporated herein by reference.

### FIELD OF THE INVENTION

The present invention generally relates to a device and method for immobilizing an in-vivo device having, for example, image and analysis transmitting capabilities. Specifically, the invention relates to a device and method for providing images or any other physiological information like temperature or pressure of an in vivo site during in vivo procedures, such as laparoscopy or Natural Opening Transgastric Endoscopy (NOTES).

### BACKGROUND OF THE INVENTION

Laparoscopy is a surgical procedure in which a special designed scope and other surgical tools are inserted into the abdomen through a small incision. It is used for a variety of surgical procedures often for bladder, prostate, small intestine as well as fallopian tubes and pelvic cavity diagnosis and surgery. Laparoscopy requires direct visualization of the peritoneal cavity, ovaries, outside of the tubes and uterus. During a typical procedure, carbon dioxide (CO₂) is put into the abdomen or other lumens through a special needle that is inserted from the out side. This gas helps in the initial separation of the organs inside the abdominal cavity, this procedure is followed by an insertion of a trocar which is a hollowed tube with an inside diameter of 5-12 mm through which the surgeon can insert his tools. Typically, the first procedure is CO₂ pumping making it easier for the physician to see organs during laparoscopy. The gas is removed at the end of the procedure.

Typically, three types of instruments called laparoscopes are used for visualization. The most common one is built like a telescope with a series of lenses and a light source. The other type is based on a bundle of optic fibers which bring light into the abdomen and carries the image outside. In some cases, an image sensor (e.g. CCD or CMOS) is attached to the tip of a laparoscope that is inserted through the trocar into the body lumen created by the CO₂ such that images of the body lumen can be displayed. The tip of sensor based laparoscope may be bendable to enable a larger field of view.

Laparoscopy may include several incisions in the abdomen. In each of them a trocar is installed through which typically a variety of surgical or therapeutic tools are inserted (such as knifes, graspers, staplers etc.) but only one incision through which to visualize the surgical site. Although a bendable scope may enable a wide field of view, it still suffers from limited angles of view and limited camera maneuvering capabilities; it does not enable viewing behind a fold or on both sides of an organ. This may be important, for example, for insertion of a needle from one side taking it out from the other side during stitching. Additionally, the use of one imager suffers, inter alia, from the fact that in order to see details the camera must zoom in on a site, in which case the orientation for the surgeon is lost. Keeping orientation may be at the expense of being able to zoom in on details, in addition using imaging devices like CCD is associated with the loss of depth orientation.

Natural Opening Transgastric Endoscopy (NOTES) is a surgical procedure in which a special designed Endoscope is inserted through the mouth into the abdomen. Then through a small incision in the stomach, the surgeon can reach inner organs such as the liver, and operate on them. The NOTES procedure eliminates cutting through muscle tissues as necessary in laparoscopy in order to reach the abdomen from outside the body. The surgical tools are inserted through a working channel of the Endoscope. As far as angles and field of view the situation is worse than laparoscopy, as the illumination device and imager are in the same direction as the tools and maneuvering capabilities are very limited.

There is therefore a great need in the art for a device and method for increasing the viewing capabilities of the surgeon when performing laparoscopy or NOTES.

Accordingly, there is now provided with this invention an improved device and method for effectively overcoming the aforementioned difficulties and longstanding problems inherent in performing surgical procedures having a limited viewing capacity.

### SUMMARY OF EMBODIMENTS OF THE INVENTION

According to one embodiment of the invention, a system for illuminating an in vivo site including an in vivo device is disclosed. According to one embodiment, the in vivo device includes a housing which contains an illumination device and a rotatable connection unit. According to one embodiment, the rotatable connection unit rotates the housing such that the illumination device may be directed to a desired spot within a body lumen.

According to one embodiment, the housing may also include a sensor, an imager, an optical system, an optical window, and a transmitter.

According to one embodiment, the housing may be capsule shaped or of any shape and size such as spherical, oval, cylindrical, etc. or other suitable shapes suitable for being inserted into the body lumen.

In another embodiment, the housing comprises a first portion and a second portion. According to one embodiment, at least one of the portions of the housing includes an illumination device. According to one embodiment, the housing may further include a rotatable connection unit, which rotates the first portion in respect to the second portion within a body lumen. The rotatable connection unit may be located in the middle of both portions, or at any other location within the housing. Typically, the two portions are facing opposing directions. According to one embodiment, when the rotatable connection unit rotates the two portions, the angle created between them is lesser than 180 degrees (as is when they are at opposing directions). According to another embodiment, the rotatable connection unit rotates an axis of said first portion in respect to an axis of a direction of imaging at an angle α, wherein 0°<α≦180°. According to one embodiment, the rotatable connection unit disclosed is preferably a ball-and-socket joint connection unit.

According to one embodiment, in addition to an illumination device, both portions may further comprise at least one imager, a sensor, an optical system, an optical window, and a transmitter. The housing, whether comprises one portion or more, may be inserted into the body lumen through a trocar, endoscope, laparoscope etc.

According to another embodiment, the in vivo device includes means for immobilization of the housing to the body lumen. For example, the device may include a first shaft housed within a second shaft. These shafts may be connected to the housing through the rotatable connection unit. The first shaft is configured to rotate the device, while the second shaft is configured for immobilizing the device to a desired spot within a body lumen. The device may instead be immobilized to an instrument inserted into the body lumen, such as a: knife, scissor, grasper, stitcher, trocar tube, endoscope, laparoscope, needle, catheter, overtube, and Percutaneous Endoscopic Gastrostomy tube.

According to another embodiment, the in vivo device may include a mount. According to one embodiment, the mount contains a first arm and a second arm adapted to moving between a first position and a second position. The second position is for immobilizing the device. The device may also contain a drive mechanism for driving the arms between the first position and the second position, so when the arms are in the second position they may grasp onto a desired location within the body lumen. The drive mechanism may drive each arm independently or drive both arms synchronously. According to one embodiment, the arms may include two pins. The first pin having an axis around which the first arm rotates, and a second pin having an axis around which the second arm rotates. According to one embodiment, the drive mechanism may engage with said arms through gears.

According to one embodiment, the in vivo device further contains a transmitter. According to another embodiment, the system includes a receiver for receiving data transmitted from the in vivo device. In yet another embodiment, the transmitter and receiver are bi-directional offering wireless redirecting of the in vivo device within the body lumen. This wireless redirecting of the in vivo device will assist the surgeon in rotating the at least one illumination device and/or imager in any direction required for better viewing during the procedure, while freeing the surgeon from attending to this task manually, during surgery.

In another embodiment, the in vivo device contains a housing, which includes an illumination device, a rotatable connection unit, and a device for immobilizing the housing to a desired location within the body lumen. According to one embodiment, the immobilizing device includes a mount for attaching to the device. The mount contains a first arm and a second arm both of which are adapted to moving between a first position and a second position, wherein the second position is for immobilizing the device. The mount may further contain a drive mechanism for driving the arms between the first and second positions.

In another embodiment, the device for immobilizing the housing includes a first shaft and a second shaft which is housed within said first shaft. The first shaft may be configured to rotate the device and the second shaft may be configured for immobilizing the device to a desired spot within a body lumen.

According to another embodiment a method for providing illumination to an in vivo site is disclosed. The method may comprise the steps of immobilizing a self powered illumination device to a body lumen, and providing illumination to an in vivo site from the illumination device. According to one embodiment, the method further includes rotating the illumination device in respect to its location in the body lumen for providing a multitude of fields of illumination.

According to one embodiment, the illumination device includes a housing containing a first portion and a second portion, wherein at least one of the portions comprises an illumination device. According to one embodiment, the housing further contains a rotatable connection unit, which rotates the first portion in respect to the second portion within a body lumen.

According to another embodiment, the immobilization is done using a mount for attaching to the illumination device. The mount may include a first arm and a second arm both of which are adapted to moving between a first position and a second position, wherein the second position is for immobilizing the illumination device. According to one embodiment, the mount may include a drive mechanism for driving the arms between the first and second positions. According to one embodiment the drive mechanism may drive each arm independently or drive both arms synchronously.

As will be appreciated by those persons skilled in the art, a major advantage provided by embodiments of the present invention is to assist a surgeon in visualizing (e.g., by illuminating) the laparoscopic procedure that he may be performing. Another advantage provided by embodiments of the present invention is the fact that trocars may be freed from the need to house visualization instruments, thereby allowing more space for surgical or other treatment tools. Other advantages may include having to perform fewer surgical incisions to achieve better visualization. In some embodiments the same area may be visualized from two different angles enabling depth orientation and three-dimensional imaging. Additional objects of the present invention will become apparent from the following description.

The device of the present invention will be better understood by reference to the following detailed discussion of specific embodiments and the attached figures which illustrate and exemplify such embodiments.

### DETAILED DESCRIPTION OF THE DRAWINGS

A specific embodiment of the present invention will be described with reference to the following drawings, wherein:

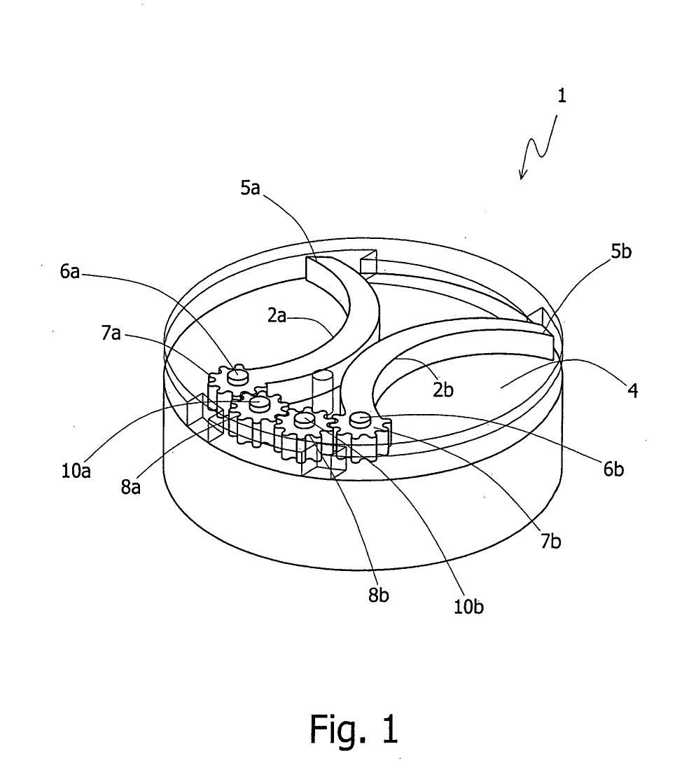

FIG. 1 is a perspective view of an embodiment of the present invention in the closed, unengaged position.

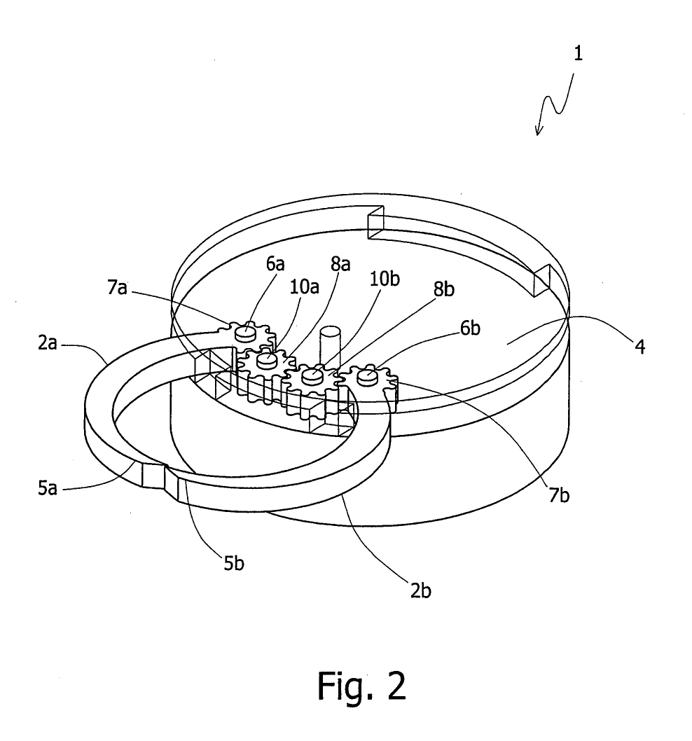

FIG. 2 is a perspective view of an embodiment of the present invention in the open, engaged position.

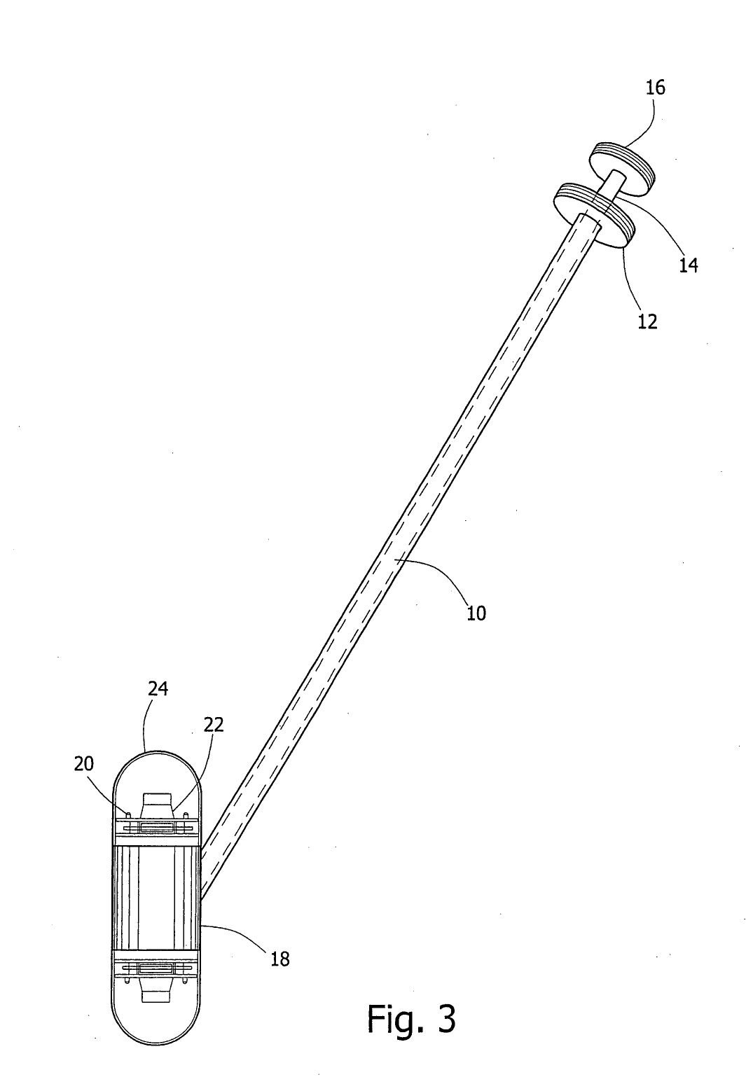

FIG. 3 is a perspective view of an embodiment of the present invention illustrating a manipulator arm attached to an in-vivo capsule.

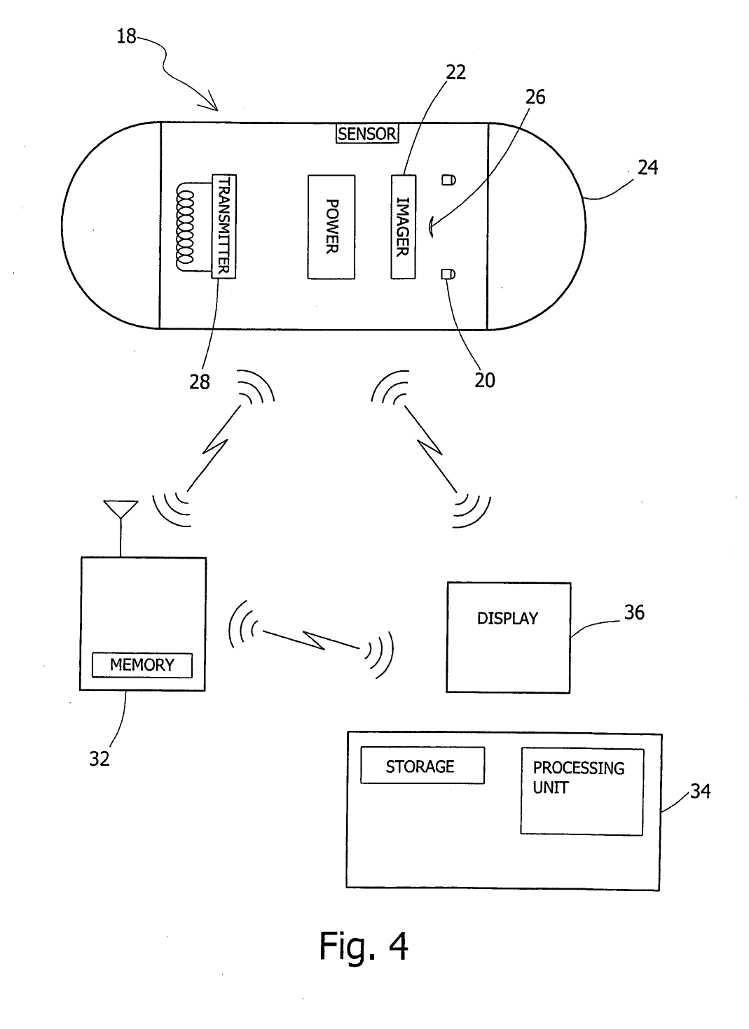

FIG. 4 is a perspective view of a capsule for transmitting images.

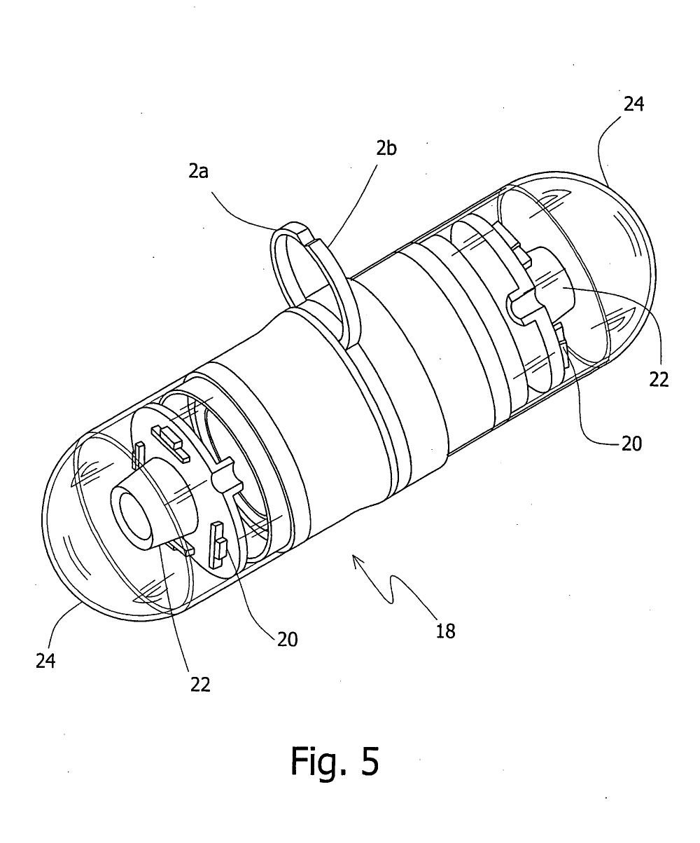

FIG. 5 is a perspective view of a capsule having an embodiment of the present invention.

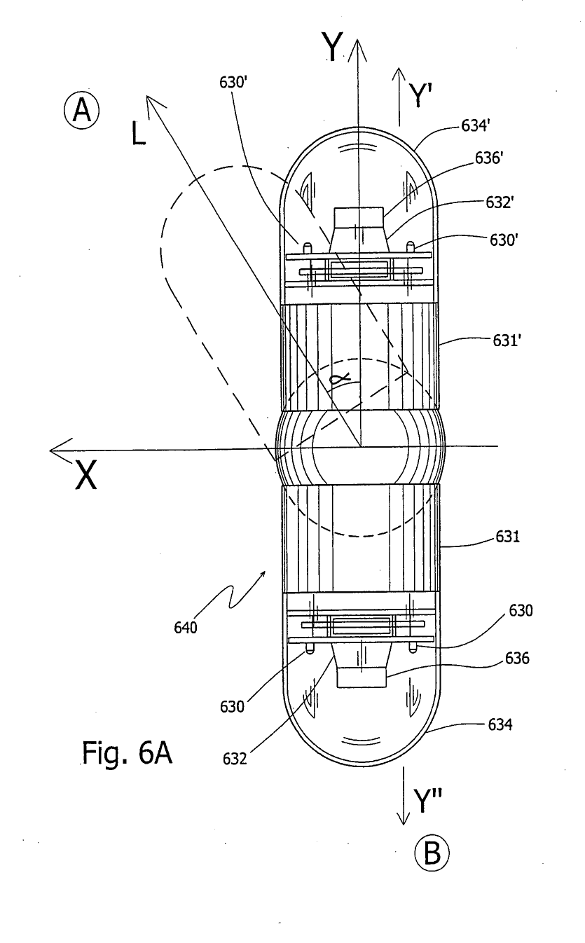

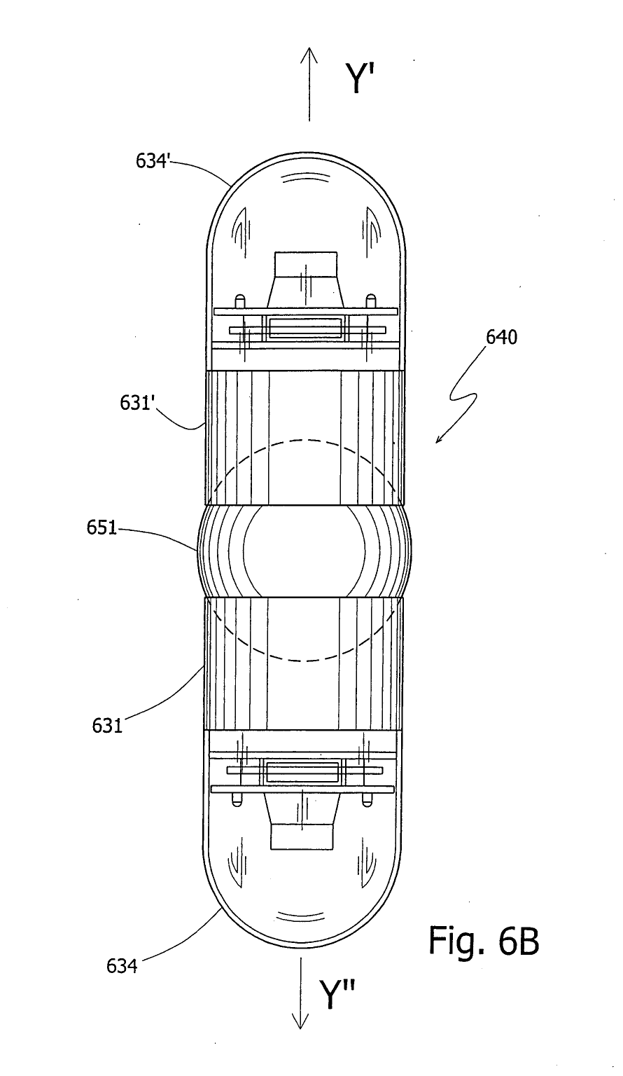

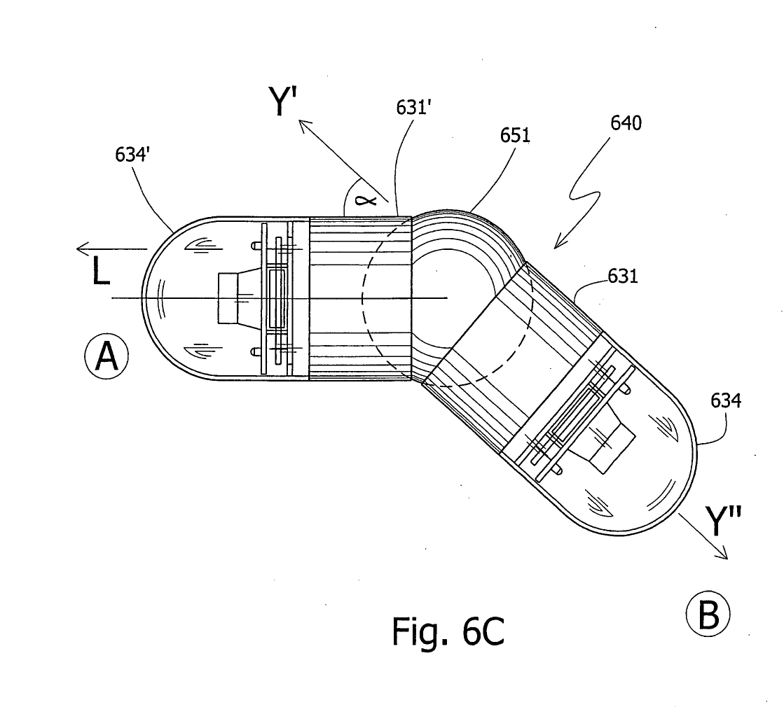

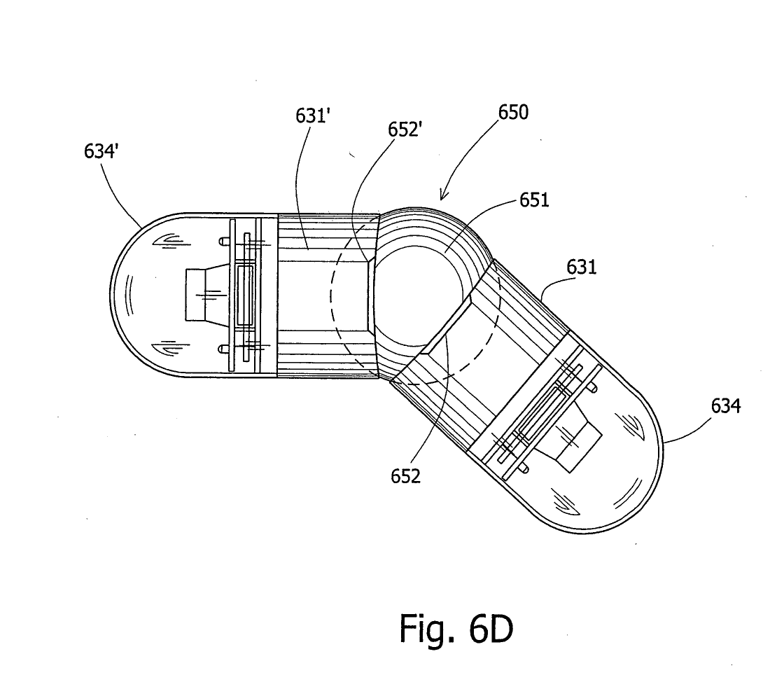

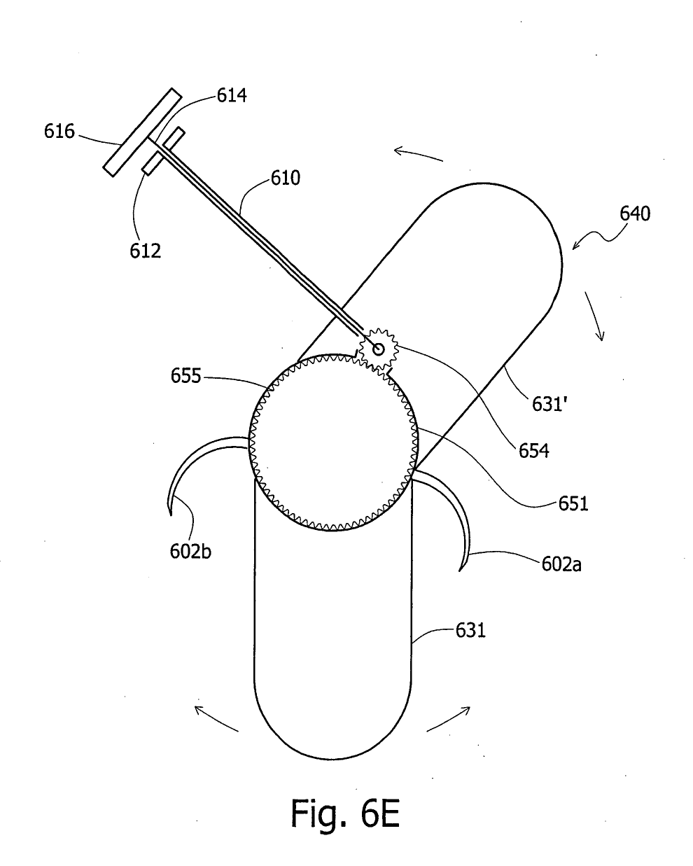

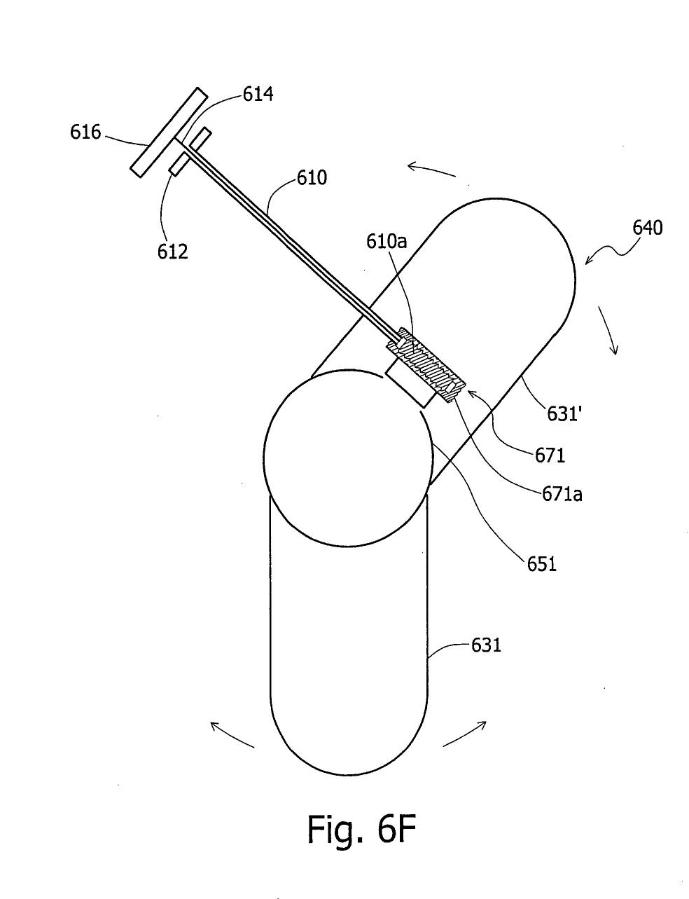

FIGS. 6A-6F are perspective views of a capsule having an embodiment of the present invention.

### DETAILED DESCRIPTION OF EMBODIMENTS OF THE INVENTION

The following preferred embodiment as exemplified by the drawings is illustrative of the invention and is not intended to limit the invention as encompassed by the claims of this application.

There is provided, in accordance with some embodiments of the present invention an in-vivo imaging system and device which may allow new angles of view in laparoscopy improving efficacy and safety of the procedure. An apparatus and method for immobilizing an in-vivo capsule is disclosed herein.

The apparatus 1, as illustrated generally in FIGS. 1 and 2, shows an embodiment of the present invention. An embodiment of the apparatus has a pair of grasping tongs or arms 2a and 2b. The grasping arms 2a and 2b may be made of a wide variety of biocompatible materials, for example, stainless steel or a rigid plastic, for example, a rigid polymer. Although the arms illustrated in this embodiment are crescent shaped, the arms or grasping tongs may take a wide variety of conformations for the purposes of the present invention. According to one embodiment, the arms may be positioned on a mount 4 and have a distal end 5a and 5b and a rotational end 7a and 7b. The rotational ends of the arms may be affixed to a mount 4 by pins 6a and 6b, respectively. The arms are adapted for pivoting about pins 6a and 6b. Also attached to the mount 4 are drive wheels 8a and 8b, which may be attached to the mount by pins 10a and 10b, respectively. The drive wheels are adapted to rotate about their respective pins e.g. pins 10a and 10b. The drive wheels 8a and 8b drive arms 2a and 2b respectively, between a closed, unengaged position illustrated in FIG. 1 and an open, grasping position illustrated in FIG. 2. The drive wheels may be made to engage their respective arms by a wide variety of methods well known to those skilled in the art, for example, by frictional drive, by intersecting gears, or the like.

The drive wheels may be made to rotate by a wide variety of methods, also well known to those skilled in the art. For example, the drive wheels may be controlled by a remote controlled motor, the armature of which may be engaged, for example, with the pins 10a and 10b. The drive wheels in this embodiment may be fixed with respect to the pins. In another embodiment of the present invention, the drive wheels may be made to rotate by an extended drive shaft that may engage with the wheels from a distance and which may be turned either by motor or manually.

As the arms 2a and 2b move from a first unengaged position towards the fully grasped position of FIG. 2, they may extend upon the walls of a body lumen and thereby immobilize the housing. In this way, the arms do not necessarily need to grasp or pinch a portion of a body between their ends 5a and 5b as illustrated in FIG. 2, but may extend apart from one another and impinge on a body lumen wall. Of course, the tongs may not only grasp and hold an in-vivo device on the body wall but may alternatively hold the in-vivo device upon anything desired by the surgeon, for example, upon another surgical tool.

The embodiments of the present invention are preferentially adapted for use with an in-vivo sensing device, for example, an in-vivo imaging capsule. When an in-vivo imaging device has an imaging end, the immobilizing device of the present invention is preferentially spaced apart from that end. For example, an embodiment of the immobilizing device of the present invention may be located at the end opposite from the imaging end of the imaging capsule. Alternatively, an embodiment of the immobilizing device of the present invention may be located in the middle portion of the capsule. In those cases where the in-vivo imaging capsule has an imaging feature on both ends, then an embodiment of the immobilizing device of the present invention may be located in between these two imaging features. According to some embodiments, an immobilizing device may include a mount which is inserted or attached to an imaging or illumination capsule. According to other embodiments, the immobilizing device may be attached to a capsule without a mount.

As shown in FIG. 3, another embodiment of the present invention may be used with an extended hollow shaft 10. A handle 12 may be fixed to shaft 10. A second shaft 14 may be housed within shaft 10. Second shaft 14 may also have a handle 16 affixed thereto. As shown in the embodiment of FIG. 3, shaft 10 may be attached to an in-vivo device, for example, a capsule 18 having illuminating capabilities 20 and/or imaging features 22. Shaft 10 may be attached to the capsule by a ball joint thereby providing a multitude of viewing angles for the surgeon and/or a multitude of fields of illumination.

A device according to one embodiment of the invention, such as a capsule 18, may be an autonomous, and possibly a single use imaging device. The imaging device 22, according to one embodiment may include an illumination source 20 such as a LED for illuminating a site in vivo, an image sensor, such as a CCD or CMOS, for imaging the site in vivo and an optical system, which may include a lens or set of lenses, for focusing the image of the in vivo site on the image sensor. Typically, the image sensor, optical system and illumination source are enclosed within a housing of the imaging device. According to one embodiment the imager, optical system and illumination source are all positioned behind a viewing window 24, which may be part of the device housing and through which the in vivo site may be illuminated and imaged. Also included within the device housing may be a wireless transmitter for transmitting image data captured by the imager and a power source, such as a battery, to power the components of the device.

According to some embodiments an imaging device may include two imagers, as shown in FIG. 3, and viewing windows, typically facing opposing directions for imaging a wider field of view.

As illustrated by an embodiment of an in-vivo imager in FIG. 4, image (and other) data transmitted from the imaging device may be received out side a patient's body by a receiver (32) placed on or near the patient's body. The data may then be transferred to a workstation (34) and may be displayed on a display (36) of the workstation for a surgeon or other professional to view during or after a procedure. In some embodiments the receiver and workstation are integrated into one unit. In yet another embodiment, the receiver (32) and transmitter (28) may be bi-directional. Receiver (32) may receive data transmitted by the transmitter (28) and send command signals to the transmitter.

An imaging device according to embodiments of the invention may be introduced into a body lumen through the trocar and may be manipulated to a desired spot and fastened to that spot by a grasper or any other appropriate tools that are inserted to the lumen either through another trocar or the same one.

As shown in FIG. 5, one embodiment of the invention includes the immobilization unit of the present invention housed within or attached to an autonomous imaging device. The embodiment of such an immobilizing unit may be used to fasten the device to a desired spot within the body lumen. According to other embodiments an autonomous imaging device may be attached to or fitted on a unit such as a grasper or a stitching device, a knife or scissor thus providing close-up view of the cut or stitch as it is being formed. According to further embodiments, an imaging device may be attached to a trocar tube from its out side without blocking the trocar opening such that a tool inserted and operated through that trocar may be viewed by the operator.

FIG. 6A is a schematic illustration of an in-vivo imaging device, e.g. in-vivo device 640, in accordance with some embodiments of the present invention. Device 640 may include elements similar to devices described above. Device 640 may include two heads, for example two transparent elongated optical heads 634 and 634′ behind which are situated illumination sources 630 and 630′, such as one or more LEDs (Light Emitting Diode), and/or OLEDs (Organic LED) or other illumination sources, two lens holders 636 and 636′, two imagers 632 and 632′ a transmitter such as an ASIC and/or a receiver and a processor. According to one embodiment, the transmitter and/or receiver of device 640 are bi-directional. The transmitter may transmit data from device 640 to the receiver and receive commands from the receiver. The device 640 may further include power source(s), which may provide power to the entirety of electrical elements of the device 640, an antenna for transmitting and receiving, for example signals from the imagers 632 and 632′. The optical head 634 may be part of housing 631, while the optical head 634′ may be part of housing 631′. The device 640 may for example simultaneously or substantially simultaneously obtain images of the body lumen, for example, the GI tract, from two ends of the device. The imagers 632 and 632′ need not operate simultaneously.

According to one embodiment of the present invention, device 640 may be a cylindrical capsule having a front end and a rear end, which is capable of passing through the entire GI tract. Nonetheless, it should be noted that device 640 may be of any shape and size suitable for being inserted into and passing through a body lumen or cavity, such as spherical, oval, cylindrical, etc. or other suitable shapes. Furthermore, device 640 or various embodiments that may include at least some components of device 640 may be attached, tied or affixed on to an instrument that is inserted into body lumens and cavities, such as, for example, on an endoscope, laparoscope, needle, catheter an overtube, a PEG (Percutaneous Endoscopic Gastrostomy) tube etc.

According to some embodiments of the present invention, a position of an in-vivo device, such as device 640 may be changed from a straight position, as shown in FIG. 6B, to a bent position, for example as shown without limitation in FIG. 6C. For example, first housing 631 and second housing 631′ may be angled at an angle other than 180 degrees for imaging one or more fields of view and/or for simultaneously imaging in-vivo spots, such as spots A and B located at opposing directions in a body lumen e.g. the stomach or the abdominal cavity. For example, the direction of imaging Y′ of optical head 634′ may be angled at an angle α, e.g. from longitudinal axis Y to axis L, to a direction of imaging L, to image, for example, spot A, while the direction of imaging Y″ of optical head 634 may coincide with the axis Y to image, for example, spot B. Any suitable angle may be created. According to some embodiments of the present invention the first housing 631 and the second housing 631′ may be 3D (three dimensions) angled or rotated. In some embodiments of the present invention a may be more than 0 degrees and smaller than 90 degrees.

According to some embodiments of the present invention, as shown in FIG. 6D, housing 631 and 631′ may be connected to a rotatable connection unit such as a ball-and-socket joint connection unit 650 or an ‘accordion shaped’ connection unit, thus enabling bending or rotating heads 634 and 634′. The ball-and-socket joint connection unit may include a ball 651, a socket and one or more joints, such joints 652 and 652′. The bottom of housing 631 may be connected to joint 652 and the bottom of housing 631′ may be connected to joint 652′. In some embodiments of the present invention, the ball 651 may be made to rotate by an extended drive shaft that may engage the ball 651 from a distance and which may be turned either by motor or manually.

According to some embodiments of the present invention, as shown in FIG. 6E, the device 640 may be used with a first extended hollow shaft 610. A handle 612 may be fixed to shaft 610. A second shaft 614 may be housed within shaft 610. Second shaft 614 may also have a handle 616 affixed thereto. The shaft 610 may be attached to the device 640. The edge of shaft 610 may be connected to rotating ball 651 (this embodiment is not shown in the figure) and rotate it or otherwise control the angle of the different parts of device 640 e.g. the first housing 631 and the second housing 631′. For example a physician may maneuver or angle housing 631 and 631′ to image an in-vivo site, e.g. a surgical site, by rotating handle 612. According to another embodiment of the present invention, the second shaft 614 may be used to fasten the device 640 to a desired spot within the body lumen. For example, the edge of shaft 614 may be engaged with a cogwheel 654 to rotate an immobilizing unit 655 e.g. with drive wheels 8a and 8b to rotate grasping arms 2a and 2b (shown in FIGS. 1 and 2. In FIG. 6E grasping arms are shown as 602a and 602b). In yet another embodiment handle 612 may be used for maneuvering while handle 614 may be used for zooming.

According to some embodiments of the present invention, as shown in FIG. 6F, a screw such as locking screw 671, or another fixing device, such as a pin, may be used to lock the in vivo imaging device in a folded or bent position (as shown for example in FIG. 6C) or a straight position (as shown in FIG. 6B). According to some embodiments the locking screw 671 may be rotated, for example by a shaft, such as shaft 610 or an endoscope. For example, the edge of shaft 610 may be engaged with a worm wheel 610a to rotate the edge of locking screw 671 which may engage a cogwheel 671a.

As shown in FIG. 4 and FIG. 6E, some embodiments of the invention may include the immobilization unit housed within or attached to an autonomous imaging device. Such an immobilizing unit may be used to fasten the device to a desired spot within the body lumen. According to other embodiments an autonomous imaging device may be attached to or fitted on a unit such as a clamp. The clamp may then be attached to a tool such as a knife or scissor or a grasper or a stitcher, thus providing close-up view of the cut or stitch as it is being formed. According to further embodiments, an imaging device may be attached to a trocar tube from its outside without blocking the trocar opening such that a tool inserted and operated through that trocar may be viewed by the operator.

According to yet another embodiment of the present invention, device 640 may be a cylindrical capsule or any shape and size such as spherical, oval, cylindrical, etc. or other suitable shapes suitable for being inserted into and passing through the trocar. The device 640 may be inserted into the abdomen, and positioned in the desired location. Once in place, extended hollow shaft 10 (e.g. FIG. 3) having a sharp needle like edge is inserted from the out side and may lock into device 640, providing the surgeon an easy way to maneuver the imager and zooming from the outside.

Although the particular embodiments shown and described above will prove to be useful in many applications in the in-vivo imaging art and the laparoscopy art to which the present invention pertains, further modifications of the present invention will occur to persons skilled in the art. All such modifications are deemed to be within the scope and spirit of the present invention as defined by the appended claims.

What is claimed is:

1. A system for illuminating an in vivo site, said system comprising an in vivo device, said in vivo device comprises a housing,
wherein said housing comprising:
a first portion and a second portion, wherein at least one of said first portion or said second portion comprises an illumination device; and
a rotatable connection unit,
whereby said rotatable connection unit rotates said first portion in respect to said second portion within a body lumen.

2. The system according to claim 1, wherein said housing comprises an optical window.

3. The system according to claim 1, wherein said device comprises a sensor.

4. The system according to claim 1, wherein said device comprises an imager.

5. The system according to claim 1, wherein said housing encloses at least one imager, an optical system and a transmitter.

6. The system according to claim 1, wherein said housing is capsule shaped.

7. The system according to claim 1, wherein said first portion and said second portion each comprise at least one illumination source, at least one imager and an optical system.

8. The system according to claim 1, wherein said first portion and said second portion face opposing directions.

9. The system according to claim 1, wherein said rotatable connection unit is located between said first portion and said second portion.

10. The system according to claim 1, wherein said rotatable connection unit rotates an axis of said first portion in respect to an axis of a direction of imaging at an angle α, wherein 0°<α≦180°.

11. The system according to claim 1, wherein said rotatable connection unit is a ball-and-socket joint connection unit.

12. The system according to claim 1, wherein said device comprises a first shaft and a second shaft, wherein said second shaft is housed within said first shaft.

13. The system according to claim 12, wherein said first shaft is connected to said rotatable connection unit.

14. The system according to claim 12, wherein said second shaft is configured for immobilizing the device to a desired spot within a body lumen.

15. The system according to claim 12, wherein said first shaft is configured to rotate the device.

16. The system according to claim 1, wherein said device is configured for being immobilized to a body lumen.

17. The system according to claim 1, wherein said device is configured for being immobilized to an instrument, said instrument for being inserted into the body lumen.

18. The system according to claim 17, wherein said instrument is selected from the group consisting of: knife, scissor, grasper, stitcher, trocar tube, endoscope, laparoscope, needle, catheter, overtube, and Percutaneous Endoscopic Gastrostomy tube.

19. The system according to claim 1, further comprising:
a mount for attaching to the device;
a first arm and a second arm both of which are attached to the mount and adapted to moving between a first position and a second position, wherein said second position is for immobilizing the device; and
a drive mechanism for driving said arms between said first position and said second position.

20. The system according to claim 19, further comprising a first pin having an axis around which said first arm rotates and a second pin having an axis around which said second arm rotates.

21. The system according to claim 19, wherein said drive mechanism engages with said arms through gears.

22. The system according to claim 1, wherein said device further comprises a transmitter.

23. The system according to claim 22, further comprising a receiver for receiving data transmitted from said device.

24. An in vivo device comprising a housing, said housing comprising a first portion and a second portion, wherein at least one of said first portion or said second portion comprises an illumination device, and
(a) a rotatable connection unit, whereby said rotatable connection unit rotates said first portion in respect to said second portion within a body lumen; and
(b) a device for immobilizing said housing to a desired location within the body lumen.

25. The device according to claim 24, wherein said device for immobilizing said housing comprises a mount for attaching to the device, said mount comprises:
a first arm and a second arm both of which are adapted to moving between a first position and a second position, wherein said second position is for immobilizing the device, and
a drive mechanism for driving said arms between said first and second positions.

26. The device according to claim 24, wherein said device for immobilizing said housing comprises a first shaft and a second shaft, wherein said first shaft is configured for immobilizing the device to a desired spot within a body lumen and said second shaft, which is housed within said first shaft is configured to rotate the device.

27. A method for providing illumination to an in vivo site, the method comprising the steps of:
immobilizing a self powered illumination device to a body lumen; and
providing illumination to an in vivo site from said illumination device.

28. The method of claim 27 further comprising rotating said illumination device in respect to its location in the body lumen for providing a multitude of fields of illumination.

29. The method of claim 27 wherein the illumination device comprises a housing said housing comprising:
a first portion and a second portion, wherein at least one of said first portion or said second portion comprises an illumination device; and
a rotatable connection unit,
whereby said rotatable connection unit rotates said first portion in respect to said second portion within a body lumen.

30. The method of claim 27 wherein the step of immobilizing is done using a mount for attaching to the illumination device, said mount comprising:
a first arm and a second arm both of which are adapted to moving between a first position and a second position, wherein said second position is for immobilizing the illumination device, and
a drive mechanism for driving said arms between said first and second positions.

31. The method of claim 30 wherein said drive mechanism drives said arms synchronously.
